(19) United States
(12) Patent Application Publication
(10) Pub. No.: US 2024/0398610 A1
(43) Pub. Date: Dec. 5, 2024

(54) BRUXISM RELIEVING DEVICE AND BRUXISM MANAGEMENT SYSTEM

(71) Applicant: DiWAVE Inc., Seoul (KR)

(72) Inventor: Keoncheol WOO, Seoul (KR)

(21) Appl. No.: 18/694,742

(22) PCT Filed: Sep. 23, 2022
(86) PCT No.: PCT/KR2022/014246

§ 371 Date: Mar. 22, 2024

(30) Foreign Application Priority Data
Sep. 23, 2021 (KR) 10-2021-0125775
Feb. 9, 2022 (KR) 10-2022-0017059

(51) Int. Cl. A61F 5/56; A61B 5/22

(52) U.S. Cl. CPC A61F 5/566; A61B 5/22; A61F 2005/563

(57) ABSTRACT

The present invention provides a bruxism relieving device and a bruxism management system. The bruxism relieving device is a device for relieving bruxism that occurs during sleep, and comprises: a mounting module that, when the bruxism occurs, is installed in the oral cavity to sense an oral state to relieve the bruxism; and a control module provided in the mounting module to control the mounting module step by step to alleviate the bruxism on the basis of oral information sensed by the mounting module.

### TECHNICAL FIELD

The present disclosure relate to a bruxism relieving device and a bruxism management system and, more particularly, to a bruxism relieving device and a bruxism management system, which relieve bruxism by determining whether the bruxism occurs during sleep, are continuously adapted to a user based on sensing information that is generated while a device operates in a process of the device being charged, provide information on diagnosis by identifying sleep-related breathing disorders that are accompanied by the bruxism, and relieve the bruxism.

### BACKGROUND ART

A person variously behaves without his or her own awareness during sleep, which may be universally called “sleep talking” or “growling when he or she wakes up”.

However, there are problems in that during such sleep talking, the quality of sleep is reduced and a case in which life-threatening occurs if sleep talking is excessive or a shock occurring during sleep talking is accumulated and degenerated into disorders may also occur.

In particular, in a case, such as bruxism in which a person grinds his or her upper teeth and lower teeth by squeezing the teeth hard, if the bruxism excessively occurs, the quality of sleep is reduced, and the jawbones may also be strained in a process of the bruxism occurring and a problem with the teeth may eventually occur.

For this reason, in order to solve the problems, efforts have been made to solve the problems by using various methods, such as vibration, noise, and notification, in order to determine a bruxism state through mounting on teeth or by detecting noise variously and to stop the bruxism when it is determined that a user is in the bruxism state. However, there is a difficulty in universally using the methods by substantially incorporating the methods. In particular, there is a difficulty in properly applying the methods to each person because each person grinds his or her teeth with a different degree of pressure or has different bruxism habits.

Furthermore, there is a case in which sleep disorders occur due to an apnea symptom occurring during sleep in addition to a simple bruxism state. There may be a problem in that life may be in danger in a severe case.

This is not simply the problem of the bruxism, but a significant number of patients who have bruxism are accompanied by snoring or sleep-related breathing disorders, such as a sleep apnea syndrome and an upper airway resistance syndrome. It is necessary to improve sleep apnea by checking a relation with the sleep apnea in addition to the bruxism.

Furthermore, it is the best to simultaneously solve bruxism and sleep-related breathing disorders because a problem that substantially occurs during sleep cannot be properly solved if the sleep-related breathing disorders that are accompanied by the bruxism are not solved although only the bruxism is relieved. However, it is not easy to check whether the sleep-related breathing disorders occur simultaneously with the bruxism. Furthermore, there may be some difficulties in reducing the sleep-related breathing disorders simultaneously with the relieving of the bruxism.

For this reason, various methods for relieving sleep-related breathing disorders that are accompanied by bruxism, in addition to the relieving of the bruxism having a form customized for each person, while more effectively relieving the bruxism of a user have been devised, and there is a need for means for solving the methods.

### DISCLOSURE

### Technical Problem

The present disclosure is an invention contrived to solve the problems of the conventional technologies, and has an object of relieving bruxism by determining whether the bruxism occurs during sleep, being continuously adapted to a user based on sensing information that is generated while a device operates in a process of the device being charged, providing information on diagnosis by identifying sleep-related breathing disorders that are accompanied by the bruxism, and relieving the sleep-related breathing disorders.

Technical objects of the present disclosure are not limited to the aforementioned object, and the other objects not described above may be evidently understood from the following description by those skilled in the art.

### Technical Solution

In a bruxism relieving device and bruxism management system for achieving the aforementioned object, a bruxism relieving device that relieves bruxism occurring during sleep includes a mounting module mounted within a mouth so that bruxism is relieved when the bruxism occurs and configured to sense a state of the mouth, and a control module provided in the mounting module and configured to control the mounting module step by step so that the bruxism is reduced based on oral information sensed by the mounting module.

In this case, the mounting module is provided between upper teeth and lower teeth within the mouth, and senses pressure that occurs as the upper teeth and the lower teeth come into contact with each other.

Furthermore, the mounting module includes a first sensor unit configured to sense pressure information occurring within the mouth and a second sensor unit configured to measure the intensity of light that is incident on the mounting module.

In this case, the first sensor unit includes a vibration member configured to transmit the pressure information to the control module and to generate vibration so that the bruxism is reduced based on the pressure information by the control module.

Meanwhile, the control module controls the vibration member by determining, as a bruxism state, a case in which the pressure information is determined to be preset pressure or more based on the pressure information received from the first sensor unit.

In this case, the control module generates first vibration by operating the vibration member based on the pressure information, and generates second vibration having a relatively higher number of vibrations than the first vibration when the pressure information is determined to be the preset pressure or more even through the first vibration.

Furthermore, when the pressure information is determined to be the preset pressure or more even in the state in which the second vibration has been generated, the control module raises the strength of the vibration step by step, but stops the vibration by determining a case in which the pressure information is determined to be less than the preset pressure as the stop of the bruxism.

Furthermore, the second sensor unit generates brightness information based on the intensity of light that is incident on the mounting module and transmits the generated brightness information to the control module.

In this case, the control module determines whether the mouth is in a closed state based on the brightness information transmitted by the second sensor unit, and determines that the mouth has been closed when the brightness information is determined to be first brightness or less.

At this time, when the brightness information is determined to be more than the first brightness, the control module determines whether the brightness information is second brightness or less that is relatively greater than the first brightness, and determines that the mouth has been opened and the upper teeth and the lower teeth have not come into contact with each other when the brightness information is the second brightness or less.

Furthermore, when the brightness information is determined to be more than the second brightness, the control module determines that the mounting module is in the state in which the mounting module has been separated from the mouth.

Furthermore, the mounting module further includes a fourth sensor unit configured to generate sound information by sensing a sound attributable to sleep-related breathing disorders and to transmit the generated sound information to the control module and a fifth sensor unit configured to generate saturation information by measuring oxygen saturation in order to determine whether the sleep-related breathing disorders occurs and to transmit the saturation information to the control module.

In this case, the control module determines whether the sleep-related breathing disorders occur based on the sound information and the saturation information transmitted by the fourth sensor unit and the fifth sensor unit.

Furthermore, the control module raises the strength of the vibration by controlling the vibration member when the sleep-related breathing disorders are determined to have occurred based on the sound information and the saturation information, and operates the vibration member with a maximum output when the oxygen saturation of the saturation information is a preset oxygen saturation numerical value or less.

Meanwhile, a bruxism management system includes a bruxism relieving device including a mounting module and a control module, a charging device in which the bruxism relieving device separated from the mouth is disposed and which charges the bruxism relieving device, and a server configured to collect sensing information sensed while the bruxism relieving device is mounted and to generate a setting value of the control module.

In this case, the mounting module includes a third sensor unit configured to generate time information by matching the pressure information and the brightness information measured by the first sensor unit and the second sensor unit while the mounting module is mounted within the mouth, and an elapse time from timing at which the mounting module is mounted.

In this case, the server recognizes that the bruxism relieving device is separated and charged by the charging device, and receives, from the control module, the time information in which the pressure information and brightness information of the first sensor unit and the second sensor unit have been matched with the elapse time.

Furthermore, the server determines a sleep time when the pressure information is generated from timing at which the bruxism relieving device is mounted within the mouth based on the time information transmitted by the control module and a working time that elapses after the pressure information is generated.

Furthermore, the server generates setting values of sleep times of the first sensor unit and the second sensor unit based on the sleep time and the working time so that the bruxism relieving device measures the pressure information and the brightness information after the bruxism relieving device is mounted within the mouth.

In this case, the server generates the setting values of the sleep times of the first sensor unit and the second sensor unit, and modifies an operating timing setting value at which the control module operates the mounting module by transmitting the setting values to the control module through the charging device.

In this case, the server accumulates the pressure information, the brightness information, and the time information that are transmitted by the control module, and modifies the setting value of the sleep time based on the accumulated information.

Meanwhile, the server is connected to the control module in real time in order to determine whether sleep-related breathing disorders occur based on the information transmitted by the fourth sensor unit and fifth sensor unit of the bruxism relieving device while operating in conjunction with the bruxism relieving device.

In this case, the server determines whether the sleep-related breathing disorders occur based on the sound information and the saturation information that are transmitted by the fourth sensor unit and the fifth sensor unit, and transmits a warning signal so that an alarm is generated in order to reduce the sleep-related breathing disorders when it is determined that the sleep-related breathing disorders continue even although the control module has controlled the vibration member with a maximum output.

Furthermore, the server performs a generation of at least any one of a warning sound, text, vibration, and an alarm by a user terminal of the bruxism relieving device in order to reduce the sleep-related breathing disorders through the warning signal.

Furthermore, the server notifies the outside of a danger of a wearer by transmitting location information to the outside along with the warning signal if sleep-related breathing disorders of the wearer continues and the wearer is determined to be in a dangerous state even though he warning signal has been used for reducing the sleep-related breathing disorders of the wearer.

### Advantageous Effects

The bruxism relieving device and bruxism management system of the present disclosure for solving the above object may have effects in that they relieve bruxism by determining whether the bruxism occurs during sleep, are continuously adapted to a user based on sensing information that is generated while a device operates in a process of the device being charged, provide information on diagnosis by identifying sleep-related breathing disorders that are accompanied by the bruxism, and relieve the bruxism.

Effects of the present disclosure are not limited to the aforementioned effects, and the other effects not described above may be evidently understood by those skilled in the art from the claims.

### BRIEF DESCRIPTION OF DRAWINGS

The summary described above in addition to the detailed description of preferred embodiments of this application described below will be better understood when read in relation to the accompanying drawings.

Preferred embodiments are illustrated in the drawings for the purpose of exemplifying the present disclosure.

However, it is to be understood that this application is not limited to accurate arrangements and means.

FIG. 1 is a diagram illustrated for an overall description of a bruxism relieving device and a bruxism management system according to an embodiment of the present disclosure;

FIG. 2 is a diagram illustrated to describe the mounting state of the bruxism relieving device in the bruxism relieving device and the bruxism management system according to an embodiment of the present disclosure;

FIG. 3 is a diagram illustrated to describe the state in which the bruxism relieving device is mounted on teeth in the bruxism relieving device and the bruxism management system according to an embodiment of the present disclosure;

FIG. 4 is a diagram illustrated to describe a relation between the bruxism relieving device and a charging device in the bruxism relieving device and the bruxism management system according to an embodiment of the present disclosure;

FIG. 5 is a diagram illustrated to describe measuring processes of a first sensor unit and a second sensor unit in the bruxism relieving device and the bruxism management system according to an embodiment of the present disclosure;

FIG. 6 is a diagram illustrated to describe a third sensor unit in the bruxism relieving device and the bruxism management system according to an embodiment of the present disclosure;

FIG. 7 is a diagram illustrated to describe a control module that is managed by a server in the bruxism relieving device and the bruxism management system according to an embodiment of the present disclosure;

FIG. 8 is a diagram illustrated to describe an overall situation and example of the bruxism relieving device and the bruxism management system according to an embodiment of the present disclosure;

FIG. 9 is a diagram illustrated to describe a fourth sensor unit and a fifth sensor unit for determining sleep-related breathing disorders in the bruxism relieving device and the bruxism management system according to an embodiment of the present disclosure; and

FIG. 10 is a diagram illustrated to describe an example of locations of the fourth sensor unit and the fifth sensor unit in the bruxism relieving device and the bruxism management system according to an embodiment of the present disclosure.

### DESCRIPTION OF REFERENCE NUMERALS

S: sleep time
W: working time
100: bruxism relieving device
120: mounting module
122: first sensor unit
124: second sensor unit
126: third sensor unit
128: fourth sensor unit
129: fifth sensor unit
140: control module
200: charging device
300: server

### MODE FOR INVENTION

Hereinafter, preferred embodiments of the present disclosure by which the object of the present disclosure may be realized in detail are described with reference to the accompanying drawings.

In describing the present embodiment, the sane mane and the same reference numeral are used for the same component, and an addition description of the same component will be omitted.

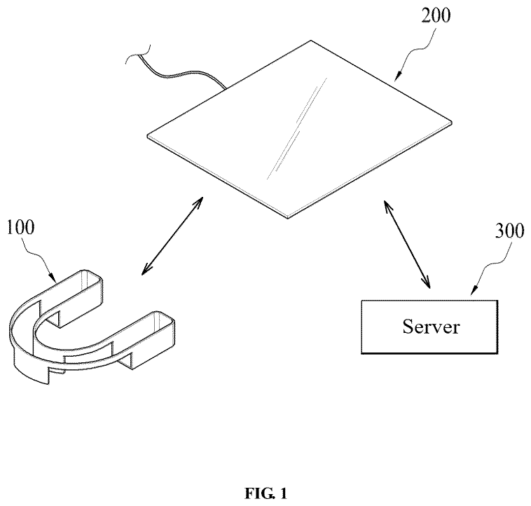

First, a bruxism relieving device and a bruxism management system according to an embodiment of the present disclosure may be generally described through FIG. 1.

Specifically, FIG. 1 is a diagram illustrated for an overall description of a bruxism relieving device and a bruxism management system according to an embodiment of the present disclosure.

First, as illustrated in FIG. 1, the bruxism relieving device and the bruxism management system according to an embodiment of the present disclosure may include a bruxism relieving device 100 including a mounting module 120 and a control module 140, a charging device 200 in which the bruxism relieving device 100 separated from the mouth is disposed and which supplements consumed power of the bruxism relieving device 100 while the bruxism relieving device 100 is mounted, and a server 300 that generates a setting value of the control module 140 by collecting sensing information from the bruxism relieving device 100.

In this case, the bruxism relieving device 100 may include the mounting module 120 that is mounted within the mouth in order to relieve bruxism when the bruxism occurs and that senses the state of the mouth and the control module 140 that is included in the mounting module 120 and that controls the mounting module 120 step by step in order to reduce bruxism based on oral information that is sensed by the mounting module 120.

In this case, the mounting module 120 is made of a silicon material, can minimize friction against teeth while bruxism occurs, and may be formed to have a preset thickness in order to prevent damage to sensors that are embedded within the mounting module to a minimum level.

Furthermore, the mounting module 120 will be more specifically described through drawings to be described in detail later, and may be mounted on the upper teeth. This may be for preventing a user from having a foreign body sensation through the tongue because the mounting module comes into contact with the bruxism relieving device 100 if the mounting module is mounted on the lower teeth.

Furthermore, the mounting module 120 is made of a silicon material, and may include various sensors therein. The mounting module is mounted within the mouth, and may be fabricated to have great durability in order to prevent the mounting module from being damaged by friction between the upper teeth and the lower teeth.

For example, a space having a form which corresponds to the lower teeth at a portion that comes into contact with the lower teeth may be formed within the mounting module 120, so that damage to the mounting module by friction against the lower teeth can be minimized. In order to protect the sensors within the mounting module against pressure that occurs as the upper teeth and the lower teeth are strongly compressed, each of portions with which the upper teeth and the lower teeth come into contact, respectively, may be formed to have a preset thickness.

Furthermore, the mounting module 120 may include a first sensor unit 122 in order to sense pressure that occurs as the upper teeth and the lower teeth are compressed in order to basically determine a bruxism state, and may include a second sensor unit 124 in order to set a working time W by determining the possibility that the upper teeth and the lower teeth may come into contact with each other in the state in which the mounting module 120 has been mounted.

This will be more specifically described later through drawings.

Meanwhile, the control module 140 may receive information from various sensor units embedded in the mounting module 120, may operate the working time W and sleep time S of the mounting module 120, etc. in accordance with a preset time zone, and may set the operating state of the mounting module 120 by using various types of sensing information.

For example, when it is determined that bruxism occurs in the state in which the mounting module 120 has been mounted within the mouth, the bruxism may be relieved by using various methods, such as that vibration is generated, notification is generated, and weak noise is generated in order to relieve the bruxism.

Meanwhile, the charging device 200 has the bruxism relieving device 100 disposed thereon, and may provide power to the bruxism relieving device 100.

In this case, the bruxism relieving device 100 may be charged with wireless power and may be charged in a wired way, but the present disclosure may not be essentially limited thereto.

Furthermore, the server 300 operates in conjunction with the control module 140 so that the server can directly receive information from the control module 140. The server may set a setting value of the control module 140 again based on information received from the control module 140, or may display, through a user's application or a separate display, information that has been generated while the bruxism relieving device 100 is mounted so that the user can check the information. This may be variously used by using a display that is provided in the charging device 200 or using a separate member.

Meanwhile, the server 300 is a software portion in general, and may mean a target that directly receives information from the control module 140. An example of the server may mean software that is embedded in a user's terminal, tablet, PC, etc.

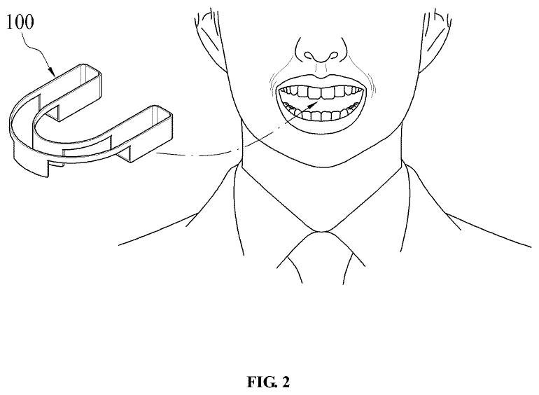

More detailed descriptions will be given with reference to FIGS. 2 to 4 based on the aforementioned approximate description.

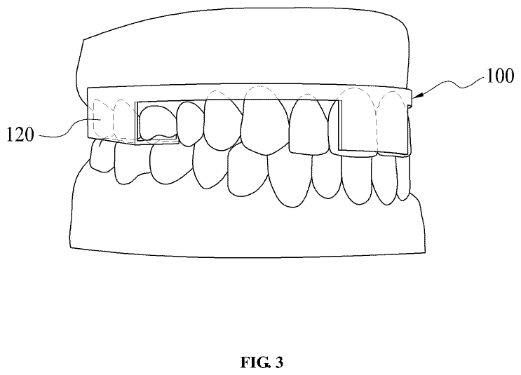

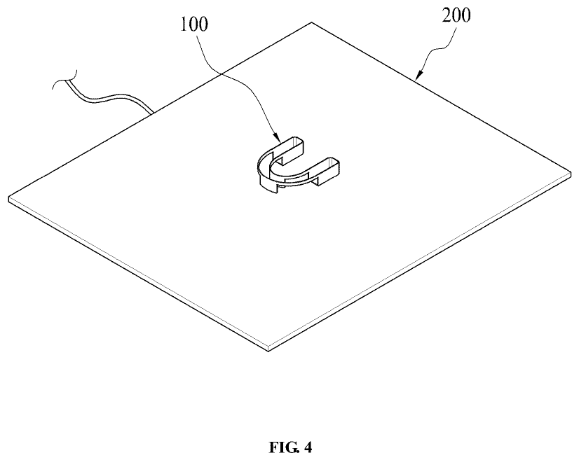

Specifically, FIG. 2 is a diagram illustrated to describe the mounting state of the bruxism relieving device in the bruxism relieving device and the bruxism management system according to an embodiment of the present disclosure. FIG. 3 is a diagram illustrated to describe the state in which the bruxism relieving device is mounted on teeth in the bruxism relieving device and the bruxism management system according to an embodiment of the present disclosure. FIG. 4 is a diagram illustrated to describe a relation between the bruxism relieving device and a charging device in the bruxism relieving device and the bruxism management system according to an embodiment of the present disclosure.

First, as illustrated in FIG. 2, the bruxism relieving device 100 may be mounted within the mouth before a user takes sleep, and may be mounted on the upper teeth in order to prevent the quality of sleep from being reduced due to the feeling of a foreign body sensation through the tongue as described above. The mounting module 120 may be mounted to grasp the upper teeth in a form in which the mounting module surrounds the upper teeth.

Furthermore, the first sensor unit 122 that senses pressure in order to sense that bruxism occurs may be mounted on a molar portion having a wide portion with which the upper teeth and the lower teeth come into contact and having a relatively wide friction area. The first sensor unit may be disposed between the molar of the upper teeth and the molar of the lower teeth.

That is, as illustrated in FIG. 3, the first sensor unit 122 may sense information that is generated as the molar of the upper teeth and the molar of the lower teeth come into contact with each other between the molar of the upper teeth and the molar of the lower teeth. The first sensor unit 122 may transmit information on generated pressure to the control module 140 so that the control module 140 determines whether bruxism occurs.

At this time, the control module 140 may receive the pressure information from the first sensor unit 122, may vibration when the bruxism state is determined based on the pressure information received from the first sensor unit 122, and may generate the vibration through a vibration member provided adjacently to the first sensor unit 122 so that the bruxism of the user can be relieved.

However, the control module 140 preferentially generates low vibration so that the vibration is not generated to the extent that the quality of sleep is broken. When the bruxism state is determined based on the pressure information, the control module 140 may generate first vibration by controlling the vibration member.

In such a condition, when the pressure that is determined based on the pressure information is determined to be preset pressure or more even though the first vibration has been generated, the control module 140 may generate second vibration corresponding to vibration higher than the first vibration by determining the user to be in the state in which the user continues the bruxism.

If the pressure is determined to be the preset pressure or more even in the state in which the second vibration has been generated and it is determined that the user is in the state in which the user consistently grinds his or her teeth, the control module may sequentially raise the strength of the vibration that is generated by the vibration member.

That is, the control module 140 consistently generates vibration by controlling the vibration member until pressure information measured by the first sensor unit 122 becomes less than the preset pressure and the bruxism is determined to be in the state in which the bruxism has been relieved, but determines the state in which the bruxism continues by determining pressure information at a preset interval, and raises the strength of the vibration step by step compared to previous vibration if the bruxism continues even after the preset interval so that the bruxism of the user can be relieved.

In general, this may have effects in that information can be transmitted to a user in various ways in order to relieve the bruxism of the user, the state in which the quality of sleep is reduced because a user wakes up from a sleep state can be prevented, and the bruxism can be relieved while maintaining the sleep state of the user because the bruxism state is checked by raising vibration from low vibration to high vibration.

Meanwhile, as illustrated in FIG. 4, if the bruxism relieving device 100 is separated because a user wakes up from the sleep state, the bruxism relieving device 100 may be charged by placing the bruxism relieving device on the charging device 200. The charging device 200 may provide power to the bruxism relieving device 100 by using a wireless charging method.

At this time, when the bruxism relieving device 100 recognizes that it is charged by the charging device 200, the bruxism relieving device 100 and the server 300 may directly operate in conjunction with each other. The server 300 may receive information transmitted by the bruxism relieving device 100.

Furthermore, the control module 140 of the bruxism relieving device 100 and the server 300 may exchange information by using short-range wireless communication, for example, Bluetooth, Wi-Fi Direct, near field communication (NFC) etc., but the present disclosure may not be essentially limited thereto.

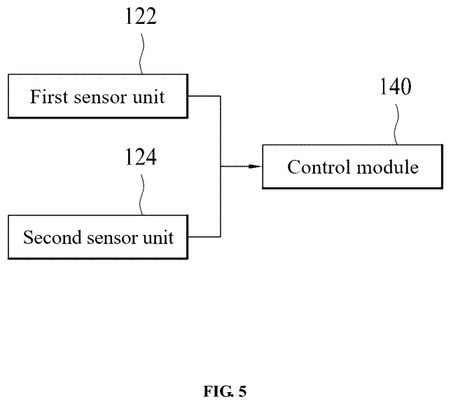

Meanwhile, reference may be made to FIGS. 5 to 8 in order to more specifically describe a process of the bruxism relieving device and the bruxism management system according to an embodiment of the present disclosure.

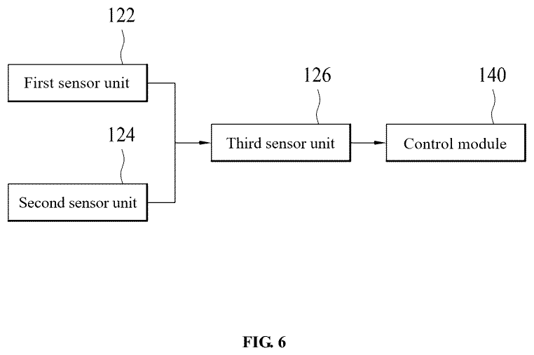

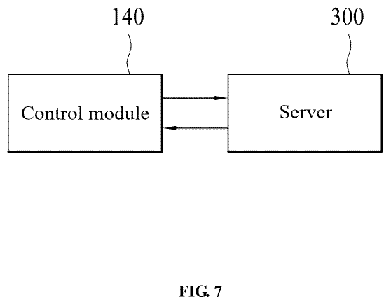

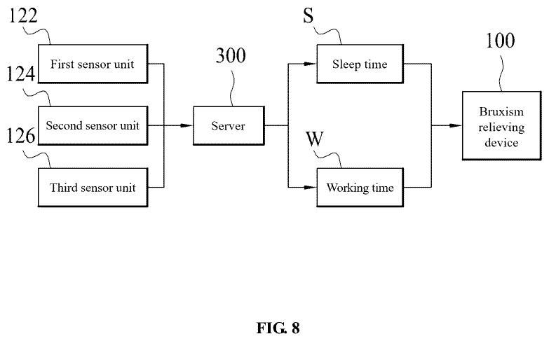

Specifically, FIG. 5 is a diagram illustrated to describe measuring processes of the first sensor unit and the second sensor unit in the bruxism relieving device and the bruxism management system according to an embodiment of the present disclosure, FIG. 6 is a diagram illustrated to describe a third sensor unit in the bruxism relieving device and the bruxism management system according to an embodiment of the present disclosure, FIG. 7 is a diagram illustrated to describe the control module that is managed by the server in the bruxism relieving device and the bruxism management system according to an embodiment of the present disclosure, and FIG. 8 is a diagram illustrated to describe an overall situation and example of the bruxism relieving device and the bruxism management system according to an embodiment of the present disclosure.

First, as illustrated in FIG. 5, the mounting module 120 may be equipped with the first sensor unit 122 and the second sensor unit 124. The first sensor unit 122 may sense pressure information as described above.

In this case, the second sensor unit 124 may detect the intensity of light that is incident on the device module so that the mounting module 120 can operate to sense pressure and the control module 140 can distinguish between the working time W when the vibration member is controlled and the sleep time S when the state of the first sensor unit 122 is temporarily changed into a power-saving state.

In view of the characteristic of the bruxism relieving device 100 that needs to be mounted within the mouth, the capacity of a battery is limited. This is for preventing the sleep state from being hindered due to a foreign body sensation because a large battery is inserted in order to increase the capacity of the battery and an area occupied by the large battery within the mouth is increased. Accordingly, there is an advantage in that sleep having high quality can be guaranteed for a user in the state in which a foreign body sensation has been minimized.

Meanwhile, the control module 140 may determine whether to operate the vibration member and also determine whether a user is in the bruxism state by controlling the mounting module 120, based on pressure information and brightness information measured by the first sensor unit 122 and the second sensor unit 124, respectively.

Furthermore, the control module 140 may include memory so that the control module collects data for the sleep state of the user during the sleep time by exchanging information with the server 300 in the state in which the control module has been disposed in the charging device 200. The control module may consistently secure the storage capacity of the memory by transmitting contents stored in the memory to the server 300 and then initializing the data for the sleep state that have been stored in the memory.

Meanwhile, as illustrated in FIG. 6, the control module 140 may autonomously control the mounting module 120 based on the brightness information, but may preset the sleep time S and the working time W by controlling a setting value based on information of the control module 140, which is transmitted to the server 300. The working time W of the mounting module 120 may be averaged by making an elapse time from timing at which the bruxism relieving device 100 has been mounted, and the pressure information and the brightness information correspond to each other.

To this end, the mounting module 120 may further include a third sensor unit 126 that generates time information by matching an elapse time from timing at which the bruxism relieving device 100 is mounted within the mouth, and the pressure information and the brightness information. The third sensor unit 126 may start an operation based on a temperature sensor or the brightness information of the second sensor unit 124 in order to determine the timing at which the mounting module 120 is mounted within the mouth.

For example, if the time when the user enters the sleep state on average is approximately 10 minutes, the working time W is set after the elapse time of approximately 10 minutes from timing at which the bruxism relieving device 100 has been mounted within the mouth based on information consistently stored in the server 300, so that the mounting module may maintain the sleep time S prior to the working time W.

Furthermore, if an average value indicating that the user starts the bruxism state in about 1 hour from the sleep state is calculated, convenience in which the bruxism relieving device 100 may be operated based on the user, such as that the working time W is set on the basis of timing at which the bruxism state starts other than a common sleep state, can also be provided.

Meanwhile, the second sensor unit 124 generates brightness information based on the intensity of light that is incident on the mounting module 120, and transmits the brightness information to the control module 140. When the brightness information is determined to be first brightness or less, the second sensor unit may operate by determining the state in which the mouth has been closed. Accordingly, the third sensor unit 126 may operate from the moment when the first brightness or less is sensed by the second sensor unit 124.

At this time, when the brightness information is determined to be more than the first brightness, the control module 140 may determine whether the brightness information is second brightness or more, which is relatively higher than the first brightness, again. When the brightness information is more than the first brightness to the second brightness or less, the control module may determine that the probability that the bruxism state may occur is low by determining light that is incident in the state in which the mouth has been opened, that is, the state in which the mouth of the user has been slightly opened. When the brightness information is determined to be more than the second brightness, the control module may determine the mouth to be in a fully opened state or a separated state, and may change the state of the mounting module 120 into the power-saving state.

Meanwhile, as illustrated in FIG. 7, information may be directly transmitted from the control module 140 of the bruxism relieving device 100 to the server 300. The server 300 may set the sleep time S and working time W of the mounting module 120 based on the received information of the control module 140, and may provide the user with information while the bruxism relieving device 100 is used so that the user may recognize a degree of bruxism.

This is represented in detail. As illustrated in FIG. 8, the first sensor unit 122 may sense the pressure information so that the bruxism state is determined. The second sensor unit 124 may sense the brightness information so that the state in which bruxism is possible is determined. The third sensor unit 126 may generate the time information obtained by matching the pressure information of the first sensor unit 122 and the brightness information of the second sensor unit 124 with an elapse time, and may transmit the time information to the control module 140.

When the time information is transmitted to the control module 140 as described above, the control module 140 controls the mounting module 120 in real time based on the time information. In the state in which the bruxism relieving device 100 is separated and disposed on the charging device 200, the control module 140 may erase data received from the first sensor unit 122, the second sensor unit 124, and the third sensor unit 126 while transmitting the data to the server 300.

At this time, the server 300 may variously determine information, such as the time when the user goes sleep on average, an elapse time when the bruxism state occurs in the sleep state, a repetition cycle of the bruxism, and duration of the bruxism, based on the received data, may calculate setting values of the sleep time S during which the mounting module 120 maintains the power-saving state and the working time W during which the mounting module 120 operates based on the information, and may transmit the setting values to the control module 140.

Furthermore, the control module 140 may receive the setting value of the sleep time S and the setting value of the working time W from the server 300, and may apply the received setting value as a changed setting value of a previous setting value. This may become more accurate as frequency and time used are increased, and whether the bruxism of the user is reduced may also be determined. Accordingly, the bruxism will be relieved more effectively.

Meanwhile, when bruxism occurs, sleep-related breathing disorders are universally accompanied by the bruxism. If sleep disorders according to common bruxism are present, when the bruxism is relieved, a case in which sleep-related breathing disorders subsequently occur may frequently occur.

For this reason, there may be somewhat a difficulty in improving the quality of sleep by simply relieving bruxism. In particular, unlike bruxism in which the quality of sleep is reduced according to simple sleep disorders, sleep-related breathing disorders may have a danger according to suffocation in a severe case. In order to solve the sleep-related breathing disorders, it is necessary to solve sleep-related breathing disorders that are accompanied after bruxism is relieved.

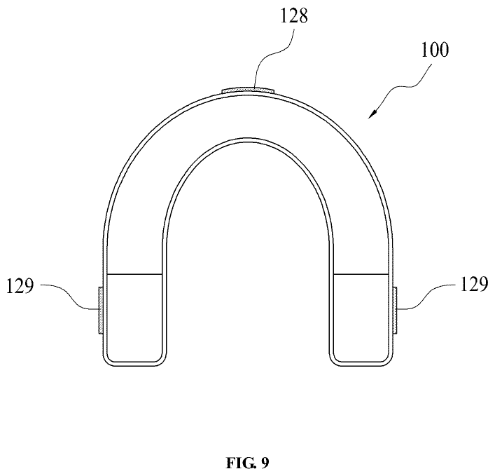

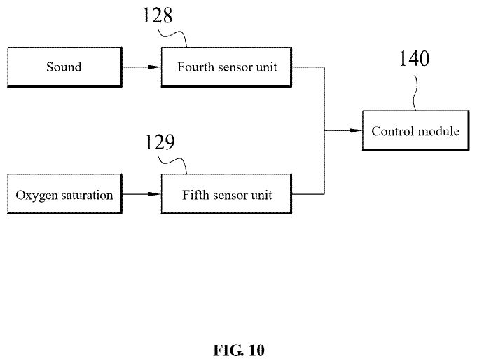

Accordingly, in order to solve sleep-related breathing disorders, a fourth sensor unit 128 and a fifth sensor unit 129 may be provided, which may be described in detail through FIGS. 9 and 10.

Specifically, FIG. 9 is a diagram illustrated to describe the fourth sensor unit and the fifth sensor unit for determining sleep-related breathing disorders in the bruxism relieving device and the bruxism management system according to an embodiment of the present disclosure. FIG. 10 is a diagram illustrated to describe an example of locations of the fourth sensor unit and the fifth sensor unit in the bruxism relieving device and the bruxism management system according to an embodiment of the present disclosure.

First, as illustrated in FIG. 9, the fourth sensor unit 128 and the fifth sensor unit 129 according to an embodiment of the present disclosure may measure a sound and oxygen saturation, respectively. The fourth sensor unit 128 may generate sound information by sensing a sound so that whether snoring occurs during sleep-related breathing disorders can be checked. The fifth sensor unit 129 may generate saturation information by measuring oxygen saturation in order to determine a sleep apnea syndrome during sleep-related breathing disorders.

In this case, the sound information generated by the fourth sensor unit 128 and the saturation information generated by the fifth sensor unit 129 may be transferred to the control module 140. The control module 140 may determine whether sleep-related breathing disorders occur based on the sound information and the saturation information.

In this case, the fourth sensor unit 128 determines the sound. If the fourth sensor unit sensitively reacts to the sound, the control module 140 may control the vibration member even though sleep-related breathing disorders have not substantially occurred. Accordingly, the fourth sensor unit may be disposed at a relatively front position on the basis of the mounting module 120. The mounting module 120 may be disposed at the front teeth in the state in which the mounting module is mounted within the mouth.

Furthermore, the fifth sensor unit 129 may be provided as a single, and may be provided as a pair. Each fifth sensor unit is disposed in at the position of the molar, but may be disposed to come into contact with the inside wall of the mouth.

This is for measuring oxygen saturation by coming into contact with the inside wall of the mouth. The fifth sensor unit may measure oxygen saturation more effectively because the fifth sensor unit comes into direct contact with the inside wall of the mouth, may generate saturation information based on the measured oxygen saturation, and may transmit the saturation information to the control module 140.

At this time, the control module 140 may determine bruxism based on the first sensor unit 122, the second sensor unit 124, and the third sensor unit 126, and may determine whether sleep-related breathing disorders occur based on information of the fourth sensor unit 128 and the fifth sensor unit 129.

If it is determined that sleep-related breathing disorders have occurred based on the sound information and the saturation information transmitted by the fourth sensor unit 128 and the fifth sensor unit 129, unlike in the previous bruxism, the vibration member may operate with a maximum output in order to reduce the sleep-related breathing disorders.

Common bruxism has a problem with the quality of sleep, whereas the sleep-related breathing disorders have a danger that may result in suffocation. Accordingly, sleep-related breathing disorders can be relieved by operating the vibration member with a maximum output as described above.

Furthermore, if it is determined that the sleep-related breathing disorders have not been relieved even though the control module 140 has operated the vibration member with a maximum output, that is, if it is determined that the sleep-related breathing disorders continue based on the sound information and the saturation information, a warning signal may be generated. The warning signal may be transmitted to the user in various ways.

For example, if the charging device 200 is disposed at a position adjacent to a user, a method of relieving sleep-related breathing disorders by generating a warning sound or generating vibration or an alarm through a separate sound generator provided in the charging device 200 or a method of reducing sleep-related breathing disorders by generating a warning sound, text, vibration, noise, etc. through a terminal that is being used by a user may also be performed. At least one of the methods may be performed.

That is, the server 300 determines whether the sleep-related breathing disorders have occurred in the user. If the sleep-related breathing disorders have occurred in the user, the server primarily attempts to reduce the sleep-related breathing disorders with a maximum output through the vibration member in order to relieve the sleep-related breathing disorders. If the sleep-related breathing disorders are not relieved nevertheless, as described above, the server 300 may secondarily perform notification, the generation of a warning sound or a sound, etc. through a device, such as a terminal, a tablet, or a PC that accompanies the server.

However, if the sleep-related breathing disorders continue despite the secondary attempt, this means that the sleep-related breathing disorders continue. Accordingly, the server 300 may determine whether the saturation information drops to a danger level, may transmit a warning signal to the outside for the safety of the user, and may also transmit location information to as an adjacent ambulance, hospital, or medical institution, in order to secure the safety of the user while transmitting the warning signal to the outside.

Furthermore, the information transmitted by the fourth sensor unit 128 and the fifth sensor unit 129 is used to determine whether sleep-related breathing disorders have occurred. Since sleep-related breathing disorders may become dangerous if they are not immediately relieved as described above, the information may operate in conjunction with the server 300 through the control module 140 so that the sleep-related breathing disorders may be determined and relieved in real time. The server 300 may be associated with the control module 140 wirelessly because the server is present on a network.

Sleep disorders can be relieved more effectively through the fourth sensor unit 128 and the fifth sensor unit 129 so that sleep-related breathing disorders accompanied by bruxism are relieved while effectively relieving the bruxism through the first sensor unit 122, the second sensor unit 124, and the third sensor unit 126 as described above.

The preferred embodiments according to the present disclosure have been described. It will be apparent to those having ordinary knowledge in the art that the present disclosure may be embodied in other specific forms without departing from the object or category of the present disclosure in addition to the aforementioned embodiments.

Accordingly, the aforementioned embodiments should not be construed as being restrictive, but should be construed as being illustrative. Accordingly, the present disclosure is not limited to the above description, and may be changed within the category of the appended claims and an equivalent range thereof.

1. A bruxism relieving device that relieves bruxism occurring during sleep, comprising:
a mounting module mounted within a mouth so that bruxism is relieved when the bruxism occurs and configured to sense a state of the mouth; and
a control module provided in the mounting module and configured to control the mounting module step by step so that the bruxism is reduced based on oral information sensed by the mounting module.

2. The bruxism relieving device according to claim 1, wherein the mounting module is provided between upper teeth and lower teeth within the mouth, and senses pressure that occurs as the upper teeth and the lower teeth come into contact with each other.

3. The bruxism relieving device according to claim 2, wherein the mounting module comprises:
a first sensor unit configured to sense pressure information occurring within the mouth; and
a second sensor unit configured to measure intensity of light that is incident on the mounting module.

4. The bruxism relieving device according to claim 3, wherein the first sensor unit comprises a vibration member configured to transmit the pressure information to the control module and to generate vibration so that the bruxism is reduced based on the pressure information by the control module.

5. The bruxism relieving device according to claim 4, wherein the control module controls the vibration member by determining, as a bruxism state, a case in which the pressure information is determined to be preset pressure or more based on the pressure information received from the first sensor unit.

6. The bruxism relieving device according to claim 5, wherein the control module generates first vibration by operating the vibration member based on the pressure information, and generates second vibration having a relatively higher number of vibrations than the first vibration when the pressure information is determined to be the preset pressure or more even through the first vibration.

7. The bruxism relieving device according to claim 6, wherein when the pressure information is determined to be the preset pressure or more even in a state in which the second vibration has been generated, the control module raises strength of the vibration step by step, but stops the vibration by determining a case in which the pressure information is determined to be less than the preset pressure as a stop of the bruxism.

8. The bruxism relieving device according to claim 3, wherein the second sensor unit generates brightness information based on an intensity of light that is incident on the mounting module and transmits the generated brightness information to the control module.

9. The bruxism relieving device according to claim 8, wherein the control module determines whether the mouth is in a closed state based on the brightness information transmitted by the second sensor unit, and determines that the mouth has been closed when the brightness information is determined to be first brightness or less.

10. The bruxism relieving device according to claim 9, wherein when the brightness information is determined to be more than the first brightness, the control module determines whether the brightness information is second brightness or less that is relatively greater than the first brightness, and determines that the mouth has been opened and the upper teeth and the lower teeth have not come into contact with each other when the brightness information is the second brightness or less.

11. The bruxism relieving device according to claim 10, wherein when the brightness information is determined to be more than the second brightness, the control module determines that the mounting module is in a state in which the mounting module has been separated from the mouth.

12. The bruxism relieving device according to claim 4, wherein the mounting module further comprises:
a fourth sensor unit configured to generate sound information by sensing a sound attributable to sleep-related breathing disorders and to transmit the generated sound information to the control module; and
a fifth sensor unit configured to generate saturation information by measuring oxygen saturation in order to determine whether the sleep-related breathing disorders occurs and to transmit the saturation information to the control module.

13. The bruxism relieving device according to claim 12, wherein the control module determines whether the sleep-related breathing disorders occur based on the sound information and the saturation information transmitted by the fourth sensor unit and the fifth sensor unit.

14. The bruxism relieving device according to claim 13, wherein the control module raises strength of the vibration by controlling the vibration member when the sleep-related breathing disorders are determined to have occurred based on the sound information and the saturation information, and operates the vibration member with a maximum output when the oxygen saturation of the saturation information is a preset oxygen saturation numerical value or less.

15. A bruxism management system that relieves bruxism occurring during sleep, the bruxism management system comprising:
the bruxism relieving device comprising the mounting module and the control module according to claim 1;
a charging device in which the bruxism relieving device separated from the mouth is disposed and which charges the bruxism relieving device; and
a server configured to collect sensing information sensed while the bruxism relieving device is mounted and to generate a setting value of the control module.

16. The bruxism management system according to claim 15, wherein the mounting module comprises a third sensor unit configured to generate time information by matching the pressure information and the brightness information measured by the first sensor unit and the second sensor unit while the mounting module is mounted within the mouth, and an elapse time from timing at which the mounting module is mounted.

17. The bruxism management system according to claim 16, wherein the server recognizes that the bruxism relieving device is separated and charged by the charging device, and receives, from the control module, the time information in which the pressure information and brightness information of the first sensor unit and the second sensor unit have been matched with the elapse time.

18. The bruxism management system according to claim 17, wherein the server determines a sleep time when the pressure information is generated from timing at which the bruxism relieving device is mounted within the mouth based on the time information transmitted by the control module and a working time that elapses after the pressure information is generated.

19. The bruxism management system according to claim 18, wherein the server generates setting values of sleep times of the first sensor unit and the second sensor unit based on the sleep time and the working time so that the bruxism relieving device measures the pressure information and the brightness information after the bruxism relieving device is mounted within the mouth.

20. The bruxism management system according to claim 19, wherein the server generates the setting values of the sleep times of the first sensor unit and the second sensor unit, and modifies an operating timing setting value at which the control module operates the mounting module by transmitting the setting values to the control module through the charging device.

21. The bruxism management system according to claim 19, wherein the server accumulates the pressure information, the brightness information, and the time information that are transmitted by the control module, and modifies the setting value of the sleep time based on the accumulated information.

22. The bruxism management system according to claim 15, wherein the server is connected to the control module in real time in order to determine whether sleep-related breathing disorders occur based on the information transmitted by the fourth sensor unit and fifth sensor unit of the bruxism relieving device while operating in conjunction with the bruxism relieving device.

23. The bruxism management system according to claim 22, wherein the server determines whether the sleep-related breathing disorders occur based on the sound information and the saturation information that are transmitted by the fourth sensor unit and the fifth sensor unit, and transmits a warning signal so that an alarm is generated in order to reduce the sleep-related breathing disorders when it is determined that the sleep-related breathing disorders continue even although the control module has controlled the vibration member with a maximum output.

24. The bruxism management system according to claim 23, wherein the server performs a generation of at least any one of a warning sound, text, vibration, and an alarm by a user terminal of the bruxism relieving device in order to reduce the sleep-related breathing disorders through the warning signal.

25. The bruxism management system according to claim 24, wherein the server notifies an outside of a danger of a wearer by transmitting location information to the outside along with the warning signal if sleep-related breathing disorders of the wearer continues and the wearer is determined to be in a dangerous state even though he warning signal has been used for reducing the sleep-related breathing disorders of the wearer.
